(19) United States
(12) Patent Application Publication
(10) Pub. No.: US 2010/0317687 A1
(43) Pub. Date: Dec. 16, 2010

(54) 2-BENZOYLIMIDAZO[1,2-a]PYRIDINE DERIVATIVES, PREPARATION THEREOF AND THERAPEUTIC USE THEREOF

(75) Inventor: Jean-Francois PEYRONEL, Palaiseau (FR)

Correspondence Address: ANDREA Q. RYAN;SANOFI-AVENTIS U.S. LLC, 1041 ROUTE 202-206, MAIL CODE: D303A, BRIDGEWATER, NJ, 08807, US

(73) Assignee: sanofi-aventis, Paris (FR)

(21) Appl. No.: 12/828,379

(22) Filed: Jul. 1, 2010

Related U.S. Application Data
(63) Continuation of application No. PCT/FR2008/001837, filed on Dec. 31, 2008.

(30) Foreign Application Priority Data
Jan. 2, 2008 (FR) 0800007

(51) Int. Cl. A61K 31/437; C07D 471/04; A61P 25/00; A61P 25/28; A61P 19/10; A61P 29/00; A61P 35/00

(52) U.S. Cl. 514/300; 546/121

(57) ABSTRACT

Compounds of formula (I)

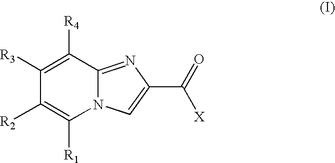

in which:
X, R₁, R₂, R₃, and R₄ are as defined in the disclosure, or an acid addition salt thereof; and therapeutic use thereof.

The present invention relates to 2-benzoylimidazo[1,2-a]pyridine derivatives, to the preparation thereof and to the therapeutic use thereof in the treatment or prevention of diseases involving Nurr-1 nuclear receptors, also known as NR4A2, NOT, TINUR, RNR-1 and HZF3.

A subject of the present invention is the compounds of formula (I):

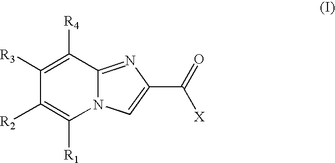

in which:
X is a phenyl group optionally substituted with one or more atoms or groups chosen, independently of one another, from the following atoms or groups: halogen, (C₁-C₆)alkyl, (C₁-C₆)alkoxy, hydroxyl, amino, NRaRb; the (C₁-C₆)alkyl and (C₁-C₆)alkoxy groups being optionally substituted with one or more halogen atoms;
R₂ is a heterocyclic group optionally substituted with one or more groups chosen, independently of one another, from the following atoms or groups: hydroxyl, halogen, (C₁-C₆)alkoxy, (C₁-C₆)alkyl optionally substituted with one or more hydroxyl, NRcRd, —CO—R₅, —CO—NR₆R₇, —CO—O—R₈, —NR₉—CO—R₁₀, cyano, and an oxido group;
R₁ is a hydrogen atom, a halogen, a (C₁-C₆)alkoxy group, a (C₁-C₆)alkyl group, hydroxyl or amino; it being possible for the (C₁-C₆)alkyl and (C₁-C₆)alkoxy groups to be optionally substituted with one or more of the following atoms or groups: halogen, hydroxyl, amino, (C₁-C₆)alkoxy;
R₃ is a hydrogen atom, a halogen atom, a (C₁-C₆)alkyl group or a hydroxyl group;
R₄ is a hydrogen atom or a halogen atom;
R₅ is a hydrogen atom or a (C₁-C₆)alkyl group;
R₆ and R₇, which may be identical or different, are a hydrogen atom or a (C₁-C₆)alkyl group, or form, with the nitrogen atom which bears them, a 4- to 7-membered ring optionally including another heteroatom chosen from N, O or S;
R₈ is a (C₁-C₆)alkyl group;
R₉ and R₁₀, which may be identical or different, are a hydrogen atom or a (C₁-C₆)alkyl group;

Ra is a (C₁-C₆)alkyl;

Rb, Rc and Rd are a hydrogen atom or a (C₁-C₆)alkyl;

in the form of a base or of an addition salt with an acid.

The compounds of formula (I) may comprise one or more asymmetrical carbon atoms. The may therefore exist in the form of enantiomers or of diastereoisomers. These enantiomers and diastereoisomers, and also mixtures thereof, including racemic mixtures, form part of the invention.

The compounds of formula (I) may exist in the form of bases or of addition salts with acids. Such addition salts form part of the invention.

These salts may be prepared with pharmaceutically acceptable acids, but the salts of other acids that are useful, for example, for purifying or isolating the compounds of formula (I) also form part of the invention.

The compounds of formula (I) may also exist in the form of hydrates or of solvates, i.e. in the form of associations or combinations with one or more water molecules or with a solvent. Such hydrates and solvates also form part of the invention.

In the context of the present invention:
the term “a halogen atom” is intended to mean: a fluorine, a chlorine, a bromine or an iodine;
the term “an alkyl group” is intended to mean: a linear, branched or cyclic, saturated aliphatic group optionally substituted with a linear, branched or cyclic, saturated alkyl group. By way of examples, mention may be made of methyl, ethyl, propyl, isopropyl, butyl, isobutyl, tert-butyl, cyclopropyl, cyclobutyl, cyclopentyl, cyclohexyl, methylcyclopropyl, etc. groups;
the term “a (C₁-C₆)alkoxy group” is intended to mean: an —O-alkyl radical where the alkyl group is as defined above;
the term “a heterocyclic group” is intended to mean: a saturated or unsaturated or partially unsaturated, monocyclic or bicyclic group containing from 5 to 10 atoms, including from 1 to 4 heteroatoms chosen from N, O and S. By way of examples of heterocyclic groups, mention may be made, in a nonlimiting manner, of: pyrrole, furan, thiophene, pyrazole, imidazole, triazole, tetrazole, oxazole, isoxazole, oxadiazole, thiazole, isothiazole, thiadiazole, pyridine, pyrimidine, pyrazine, pyridazine, triazine, furofuran, thienothiophene, pyrrolopyrrole, pyrroloimidazole, pyrrolopyrazole, pyrrolotriazole, imidazoimidazole, imidazopyrazole, furopyrrole, furoimidazole, furopyrazole, furotriazole, pyrrolo-oxazole, imidazo-oxazole, pyrazolo-oxazole, furo-oxazole, oxazolo-oxazole, oxazoloisoxazole, pyrrolo-isoxazole, imidazo-isoxazole, pyrazolo-isoxazole, isoxazolo-isoxazole, furo-isoxazole, isoxazolo-oxadiazole, pyrrolo-oxadiazole, furo-oxadiazole, isoxazolo-oxadiazole, thienopyrrole, thienoimidazole, thienopyrazole, thienotriazole, pyrrolo-thiazole, imidazo-thiazole, pyrazolo-thiazole, triazolo, thiazole, furo-thiazole, oxazolo-thiazole, oxazoloisothiazole, pyrrolo-isothiazole, imidazo-isothiazole, pyrazolo-isothiazole, isoxazolo-isothiazole, furo-isothiazole, pyrrolo-thiadiazole, imidazo-thiadiazole, furo-thiadiazole, isoxazolo-thiadiazole, oxazolo-thiadiazole, isothiazolo-thiadiazole, indole, isoindole, benzimidazole, indazole, indolizine, benzofuran, isobenzofuran, benzothiophene, benzo[c]thiophene, pyrrolopyridine, imidazopyridine, pyrazolopyridine, triazolopyridine, tetrazolopyridine, pyrrolopyrimidine, imidazopyrimidine, pyrazolopyrimidine, pyrrolopyrazine, imidazopyrazine, pyrazolopyrazine, pyrrolopyridazine, imidazopyridazine, pyrazolopyridazine, triazolopyridazine, pyrrolotriazine, furopyridine, furopyrimidine, furopyrazine, furopyridazine, furotriazine, oxazolopyridine, oxazolopyrimidine, oxazolopyrazine, oxazolopyridazine, isoxazolopyridine, isoxazolopyrimidine, isoxazolopyrazine, isoxazolopyridazine, oxadiazolopyridine, benzoxazole, benzisoxazole, benzoxadiazole, thienopyridine, thienopyrimidine, thienopyrazine, thienopyridazine, thienotriazine, thiazolopyridine, thiazolopyrimidine, thiazolopyrazine, thiazolopyridazine, isothiazolopyridine, isothiazolopyrimidine, isothiazolopyrazine, isothiazolopyridazine, thiadiazolopyridine, thiadiazolopyrimidine, benzothiazole, benzoisothiazole, benzothiadiazole, quinoline, isoquinoline, cinnoline, phthalazine, quinoxaline, quinazoline, naphthyridine, benzotriazine, pyridopyrimidine, pyridopyrazine, pyridopyridazine, pyridotriazine, pyrimidopyrimidine, pyrimidopyrazine, pyrimidopyridazine, pyrazinopyrazine, pyrazinopyridazine, pyrazinotriazine, pyridazinopyridazine; it being possible for these groups to be saturated or partially unsaturated; with the exception of the 4- to 7-membered mononitrogenous monocyclic heterocycles optionally containing another heteroatom chosen from N, S and O and linked via the nitrogen.

Among the compounds of formula (I) which are subjects of the invention, a first group of compounds is constituted of the compounds for which:

X is a phenyl group;

R₁, R₃ and R₄ are hydrogen atoms;

R₂ is an unsaturated monocyclic heterocyclic group containing 5 or 6 atoms, including from 1 to 2 heteroatoms chosen from N or O, said heterocyclic group being optionally substituted with an —NR_{c}R_{d} group, R_{c} and R_{d} being a hydrogen or a (C₁-C₆)alkyl.

Among the compounds of formula (I) which are subjects of the invention, a second group of compounds is constituted of the compounds for which:

X is a phenyl group;

R₁, R₃ and R₄ are hydrogen atoms;

R₂ is a pyridine, pyrrole, pyrazole, imidazole or furan group, optionally substituted with an NH₂ group,

in the form of a base or of an addition salt with an acid.

Among the compounds of formula (I) which are subjects of the invention, a third group of compounds is constituted of the compounds for which R₂ is a saturated or unsaturated or partially unsaturated, monocyclic or bicyclic heterocyclic group containing from 5 to 10 atoms, including from 1 to 4 heteroatoms chosen from N, O and S, with the exception of the 4- to 7-membered mononitrogenous monocyclic heterocycles optionally containing another heteroatom chosen from N, S and O and linked via the nitrogen.

Among the compounds of formula (I) which are subjects of the invention, a fourth group of compounds is constituted of the compounds for which R₂ is an unsaturated or partially saturated, monocyclic heterocyclic group containing from 5 to 7 atoms, including from 1 to 3, in particular from 1 to 2, heteroatoms chosen from O, N and S, in particular N or O.

Among the compounds of formula (I) which are subjects of the invention, a fifth group of compounds is constituted of the compounds for which R₂ is a pyrrole, furan, thiophene, pyrazole, imidazole, triazole, tetrazole, oxazole, isoxazole, oxadiazole, thiazole, isothiazole, thiadiazole, pyridine, pyrimidine, pyrazine, pyridazine or triazine group, and more particularly a pyridine, pyrrole, pyrazole, imidazole or furan group.

Among the compounds of formula (I) which are subjects of the invention, mention may in particularly be made of the following compounds:
[6-(6-aminopyridin-2-yl)imidazo[1,2-a]pyridin-2-yl](phenyl)methanone,
phenyl(6-pyridin-2-ylimidazo[1,2-a]pyridin-2-yl)methanone and the dihydrochloride thereof,
phenyl[6-(1H-pyrrol-3-yl)imidazo[1,2-a]pyridine-2-yl]methanone,
phenyl[6-(1H-pyrazol-4-yl)imidazo[1,2-a]pyridin-2-yl]methanone,
[6-(1H-imidazol-4-yl)imidazo[1,2-a]pyridin-2-yl](phenyl)methanone,
[(6-furan-2-yl)imidazo[1,2-a]pyridin-2-yl](phenyl)methanone,
phenyl[(6-pyridin-3-yl)imidazo[1,2-a]pyridin-2-yl]methanone,
   or an addition salt of these compounds with a pharmaceutically acceptable acid.

In accordance with the invention, the compounds of general formula (I) can be prepared according to the process described in scheme 1.

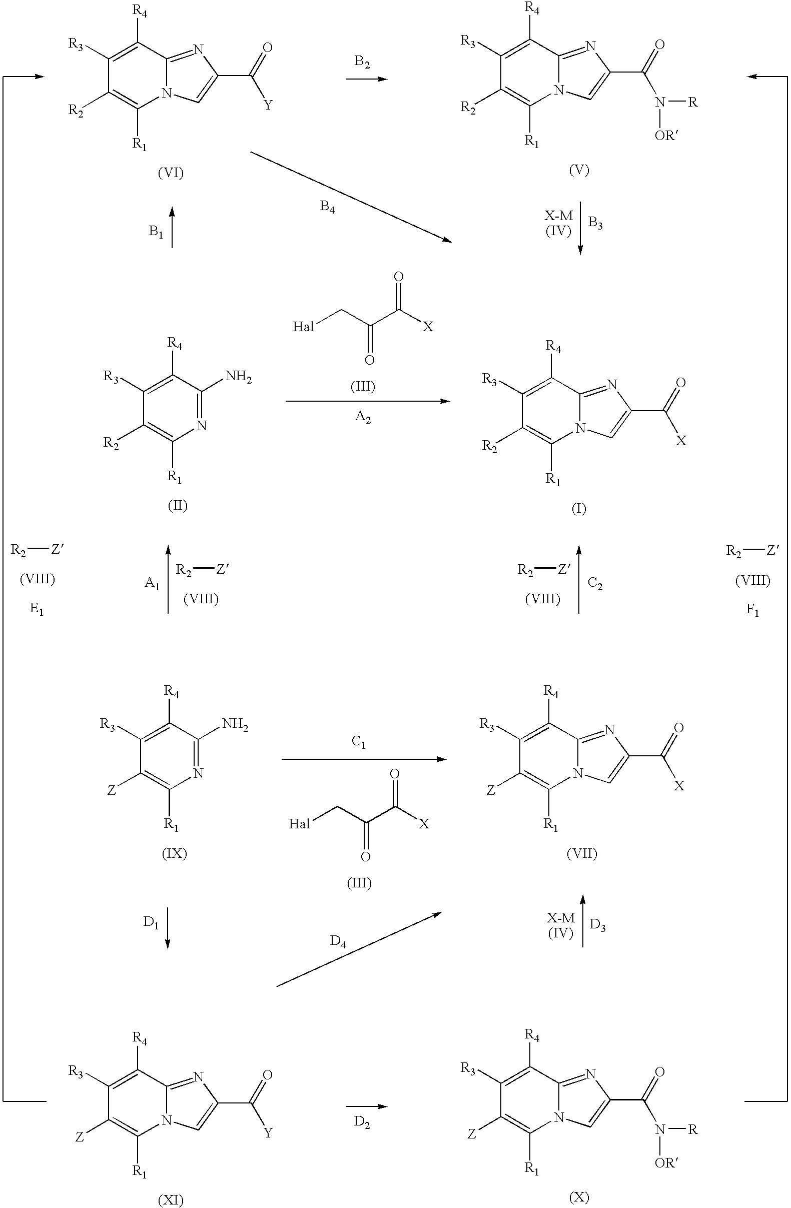

The first synthetic pathway (transformation A₂) consists in condensing a 2-aminopyridine of formula (II), in which R₁, R₂, R₃ and R₄ are as defined above, with a 3-halo-1-arylpropane-1,2-dione derivative of general formula (III), in which Hal is a chlorine, bromine or iodine atom and X is as defined above, so as to form the imidazo[1,2-a]pyridine ring, for example according to the method described by J-J. Bourguignon et al. in Aust. J. Chem., 50, 719 (1997).

The second synthetic pathway (transformation B₃ or B₄) consists in reacting an organometallic derivative of general formula (IV) in which X is as defined above and M is a lithium atom or an Mg-Hal group, with:
a Weinreb amide (or N-alkoxy-N-alkylamide) of general formula (V), in which R₁, R₂, R₃ and R₄ are as defined above and are other than bromine or iodine, and R and R′—which may be identical or different—are an alkyl group, according to methods known to those skilled in the art, as described by Weinreb, S. M. et al. in Tetrahedron Letters (1981), 22 (39), 3815-18 and in Sibi, M. P. Organic Preparations and Procedures Int. 1993, 25, 15-40 (transformation B₃), or
with an imidazo[1,2-a]pyridine-2-carboxylic acid of general formula (VI), in which R₁, R₂, R₃ and R₄ are as defined above and are other than bromine or iodine, and Y is a hydroxyl, or a reactive salt or derivative thereof, such as ester, acid halide, anhydride or amide, according to methods known to those skilled in the art, as described in J. March, Advanced Organic Chemistry (Wiley, 5th Ed. 2001) p. 567 and 1213 or in the cited references (transformation B₄).

Alternatively, transformation B₄ can be carried out by reacting a reactive derivative, such as a mixed anhydride (which can be generated in situ), of the imidazo[1,2-a]pyridine-2-carboxylic acid of formula (VI), in which Y is a hydroxyl and R₁, R₂, R₃ and R₄ are as defined above and are other than bromine or iodine, with an organometallic derivative of formula (IV), in which X is as defined above and M is a boronic group, in the presence of a palladium catalyst such as tetrakis(triphenylphosphine)palladium.

The third synthetic pathway (transformation C₂) consists in carrying out the catalytic coupling of a derivative of general formula (VII), in which R₁, R₃ and R₄ are as defined above and Z is a boryl, stannyl or silyl group, with a derivative R₂—Z′ of formula (VIII), in which Z′ is a halogen atom such as bromine or iodine or a sulphonyloxy group, and R₂ is an optionally substituted 1-alkenyl, 1-alkynyl, aryl or heteroaryl group. Alternatively, the coupling may be carried out between a derivative of general formula (VII), in which R₁, R₃ and R₄ are as defined above and Z is a halogen atom such as bromine or iodine, with a derivative R₂—Z′ (VIII), in which Z′ is a reactive group such as a boryl, stannyl or silyl group or a hydrogen atom, and R₂ is an optionally substituted 1-alkenyl, 1-alkynyl, aryl or heteroaryl group.

The 2-aminopyridines of formula (II) can be prepared according to the methods described in the literature or known to those skilled in the art. In particular, the 2-aminopyridines of formula (II), in which R₁, R₃ and R₄ are as defined above and R₂ is an optionally substituted 1-alkenyl, 1-alkynyl, aryl or heteroaryl group, can be prepared by transformation A₁, i.e. by catalytic coupling reaction,
either of a 2-aminopyridine derivative of formula (IX), in which R₁, R₃ and R₄ are as defined above and Z is a boryl, stannyl or silyl group, with a derivative R₂—Z′ (VIII), in which Z′ is a halogen atom such as bromine or iodine or a sulphonyloxy group, and R₂ is an optionally substituted 1-alkenyl, 1-alkynyl, aryl or heteroaryl group,
or of a 2-aminopyridine derivative of formula (IX), in which R₁, R₃ and R₄ are as defined above and Z is a halogen atom such as bromine or iodine, with a derivative R₂—Z′ (VIII), in which Z′ is a reactive group such as a boryl, stannyl or silyl group or a hydrogen atom, and R₂ is an optionally substituted 1-alkenyl, 1-alkynyl, aryl or heteroaryl group.

The 3-halo-1-arylpropane-1,2-dione derivatives of formula (III) can be prepared by halogenation of the corresponding 1-arylpropane-1,2-diones according to methods known to those skilled in the art.

The Weinreb amides of formula (V) can be obtained (transformation B₂) by coupling of an acid of formula (VI), in which Y is a hydroxyl group, or a reactive derivative thereof, with an N,O-dialkylamine according to methods known to those skilled in the art. The coupling may be carried out in the presence of a coupling agent such as CDI, EDCI, HATU or HBTU and of a base such as diisopropylethylamine, triethylamine or pyridine, in an inert solvent such as THF, DMF or dichloromethane. Alternatively, the N,O-dialkylamine can be reacted with an ester of formula (VI), in which Y is an alkoxy group, in the presence of a catalyst such as trimethylaluminium (Weinreb. S. M. et al., Synth. Commun. 1982, 12, 989).

The derivatives of the imidazopyridine-2-carboxylic acids of formula (VI), in which R₁, R₂, R₃ and R₄ are as defined above and Y is a (C₁-C₆)alkoxy or hydroxyl group or a halogen atom, can be prepared by condensation of a 2-aminopyridine of formula (II), in which R₁, R₂, R₃ and R₄ are as defined above, with a 3-halo-2-oxopropionic acid ester of formula (VIII), in which Hal is a chlorine, bromine or iodine atom and Y is a (C₁-C₆)alkoxy group, under the conditions described by J. G. Lombardino in J. Org. Chem., 30, 2403 (1965) for example, followed where appropriate by the conversion of the ester to acid and then to acid chloride or other reactive derivative (transformation B₁).

The imidazo[1,2-a]pyridine derivatives of formula (VII), in which X, R₁, R₃ and R₄ are as defined above and Z is a halogen atom or a boryl, stannyl or silyl group, can be prepared (transformation C₁) by condensation of a 2-aminopyridine of formula (II), in which Z, R₁, R₃ and R₄ are as defined above, with a 3-halo-1-arylpropane-1,2-dione derivative of general formula (III), in which Hal is a chlorine, bromine or iodine atom, under the conditions described above for the preparation of the products of general formula (I) via transformation A₂.

Alternatively, the imidazo[1,2-a]pyridine derivatives of formula (VII), in which X, R₁, R₃ and R₄ are as defined above and Z is a halogen atom or a boryl, stannyl or silyl group, can be prepared by reacting an organometallic derivative of general formula (IV), in which X is as defined above and M is a lithium atom or a Mg-Hal group, with an imidazo[1,2-a]pyridine-2-carboxylic acid of formula (XI), in which R₁, R₂, R₃, R₄ and Z are as defined above and are other than bromine or iodine, and Y is a hydroxyl group, or a reactive derivative thereof such as acid chloride (transformation D₄), or with a corresponding Weinreb amide of formula (X) (transformation D₃), with the other reactive functions being optionally protected, under the conditions described above for the preparation of the products of general formula (I) via transformations B₃ or B₄.

The imidazopyridine-2-carboxylic acid derivatives of formula (X) and (XI) can be prepared by condensation of a 2-aminopyridine of formula (IX), in which Z, R₁, R₃ and R₄ are as defined above, with a 3-halo-2-oxopropionic acid of formula (VIII), in which Hal is a chlorine, bromine or iodine atom and Y is a (C₁-C₆)alkoxy group, according to the methods described above for the preparation of the derivatives of formulae (V) and (VI) (transformation D₁).

The coupling of the derivatives of formula (VII), (IX) or (X) with the products of formula (VIII) can be carried out by any method known to those skilled in the art, in particular by performing the procedure in the presence of copper-based or palladium-based catalysts, of ligands such as phosphines, according to or by analogy with the methods described, for example, in the following references and cited references:
for Suzuki-type reactions: N. Miyaura, A. Suzuki, Chem. Rev., 95, 2457, (1995),
for Stille-type reactions: V. Farina et al., Org. React., 50, 1 (1997),
for Hiyama-type reactions: T. Hiyama et al., Top. Curr. Chem., 2002, 219, 61 (2002),
for Negishi-type reactions: E. Negishi et al., Chem. Rev., 103, 1979 (2003),
for Bellina-type reactions: M. Miura et al., Chem. Lett., 200 (2007).

It is also possible, in order to carry out the coupling, to intermediately form, but without isolating them, organometallic derivatives such as zinc derivatives.

In accordance with the invention, the compounds of general formulae (I), (II) and (VI) can also be prepared according to the processes described in scheme 2, i.e. by the conversion of a compound of general formula (XII), (XIII) or (XIV), in which R₁, R₃, R₄ and X are as defined above, Y is a hydroxyl, alkoxy or N-alkoxy-N-alkylamino group and W is a precursor group allowing the construction of the heterocycle of formula R₂, respectively to compounds of general formulae (I), (VI) and (II) according to the methods known to those skilled in the art (transformations G₁, G₂ and G₃).

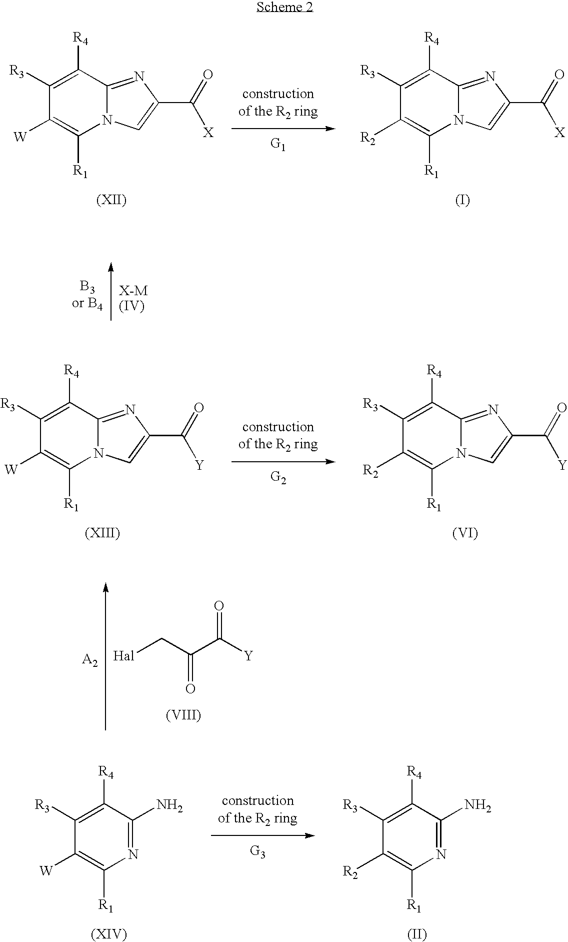

By way of example, W may be:
a 2-haloacyl group such as bromoacetyl, or a 1-halo-2-oxoalkyl group such as 1-bromo-2-oxoethyl, which can be converted, for example, to a thiazolyl, imidazolyl or oxazolyl group by treatment with thiourea derivatives, thioamide derivatives, guanidine derivatives, urea derivatives or amide derivatives;
an alkynyl group, such as ethynyl, which can be converted to a 1,2,3-triazol-4-yl group;
a cyano group which can be converted, for example, to a dihydroimidazolyl(2) or 1,3,4-triazol-2-yl group.

The compounds of general formula (XII) can be obtained from the compounds of formula (XIII), under the conditions described for the preparation of the compounds (I), from the imidazopyridine-2-carboxylic acid derivatives of formula (V) or (VI), via transformations B₂ or B₄.

The imidazopyridine-2-carboxylic acid derivatives of general formula (XIII) can be obtained from the aminopyridines of formula (XIV), under the conditions described for the conversion of the aminopyridines of formula (II) to compounds of general formula (I) via transformation A₂.

The products of formula (I) and the precursors thereof of formula (II), (V) or (VI) can be subjected, if desired and if necessary, in order to obtain products of formula (I) or to be converted to other products of formula (I), to one or more of the following transformation reactions, in any order:
a) a reaction for the esterification or amidation of an acid function,
b) a reaction for the hydrolysis of an ester function to an acid function,
c) a reaction for the transformation of a hydroxyl function to an alkoxy function,
d) a reaction for the oxidation of an alcohol function to an aldehyde or ketone function,
e) a reaction for the oxidation of an alkenyl group to an aldehyde or ketone function,
f) a reaction for the dehydration of a hydroxyalkyl group to an alkenyl group,
g) a reaction for the total or partial hydrogenation of an alkenyl or alkynyl group to an alkenyl or alkyl group,
h) a reaction for the catalytic coupling of a halogenated derivative and of an organometallic derivative, such as a stannic or boronic derivative, in order to introduce an alkyl, alkenyl, alkynyl, aryl or heteroaryl substituent,
i) a reaction for the conversion of a halogenated derivative in order to introduce a boryl, stannyl or silyl substituent,
j) a reaction for the protection of the reactive functions,
k) a reaction for the removal of the protective groups which the protected reactive functions may carry,
l) a reaction for salification with an inorganic or organic acid or with a base in order to obtain the corresponding salt,
m) a reaction for the resolution of the forms to give enantiomers, said products of formula (I) thus obtained being, where appropriate, in any of the possible racemic, enantiomeric and diastereoisomeric isomer forms.

In scheme 1, the starting compounds and the reactants, when the method for preparing them is not described, are commercially available or described in the literature or else can be prepared according to methods which are described therein or which are known to those skilled in the art.

The following examples describe the preparation of some compounds in accordance with the invention. These examples are not limiting and serve only to illustrate the present invention. The numbers of the compounds exemplified refer back to those given in the tables hereinafter, which illustrate the chemical structures and the spectroscopic characteristics of some compounds according to the invention.

### EXAMPLE 1

### [6-(6-Aminopyridin-2-yl)imidazo[1,2-a]pyridin-2-yl](phenyl)-methanone

A microwave tube is loaded with 300 mg of phenyl[6-(4,4,5,5-tetramethyl-1,3,2-dioxaborolan-2-yl)imidazo[1,2-a]pyridin-2-yl]methanone, 123 mg of 2-amino-6-bromopyridine, 30 mg of tetrakis(triphenylphosphine)palladium, 2 ml of a 2M solution of sodium carbonate, 4 ml of toluene and 4 ml of acetonitrile. The reaction mixture is heated for 20 minutes in the microwave apparatus set at 150° C., and then cooled, filtered through celite, diluted with ethyl acetate, dried over magnesium sulphate and concentrated under reduced pressure. The residue is chromatographed on a silica cartridge, elution being carried out with a mixture of dichloromethane and ethyl acetate (50/50). The fractions containing the expected product are combined and concentrated to dryness under reduced pressure. The solid is triturated in a mixture of dichloromethane and isopropyl ether and then dried, to give 57 mg of [6-(6-aminopyridin-2-yl)imidazo[1,2-a]pyridin-2-yl](phenyl)methanone in the form of a brown solid.

### EXAMPLE 2

### Phenyl[(6-pyridin-2-yl)imidazo[1,2-a]pyridin-2-yl]methanone hydrochloride (1:2)

### 2.1: Phenyl[6-(pyridin-2-yl)imidazo[1,2-a]pyridin-2-yl]methanone

A microwave tube is loaded with 200 mg of phenyl[6-(4,4,5,5-tetramethyl-1,3,2-dioxaborolan-2-yl)imidazo[1,2-a]pyridin-2-yl]methanone, 130 mg of 2-iodopyridine, 26 mg of tetrakis(triphenylphosphine)palladium, 2 ml of a 2M solution of sodium carbonate, 4 ml of toluene and 4 ml of acetonitrile. The reaction mixture is heated for 20 minutes in the microwave apparatus set at 150° C., and then cooled, filtered through celite and concentrated under reduced pressure. The residue is chromatographed on a silica cartridge, elution being carried out with a mixture of dichloromethane and ethyl acetate (75/25). The fractions containing the expected product are combined and concentrated to dryness under reduced pressure. The solid is triturated in methanol and then filtered, to give 95 mg of phenyl(6-pyridin-2-ylimidazo[1,2-a]pyridin-2-yl)methanone in the form of an ecru solid.

### 2.2: Phenyl[6-(pyridin-2-yl)imidazo[1,2-a]pyridin-2-yl]methanone hydrochloride (1:2)

200 μl of a 4M solution of hydrochloric acid in dioxane are added to a solution of 95 mg of phenyl[6-(pyridin-2-yl)imidazo[1,2-a]pyridin-2-yl]methanone in the minimum amount of dioxane. The precipitate formed is spin-filter-dried, washed with ethyl ether and dried, to give 94 mg of phenyl(6-pyridin-2-ylimidazo[1,2-a]pyridin-2-yl)methanone dihydrochloride in the form of a beige solid.

### EXAMPLE 3

### Phenyl[6-(1H-pyrrol-3-yl)imidazo pyridin-2-yl]methanone

### 3.1: Phenyl[6-(1-triisopropylsilylpyrrol-3-yl)imidazo[1,2-a]pyridin-2-yl]methanone

A microwave tube is loaded with 225 mg of (6-iodolimidazo[1,2-a]pyridin-2-yl)(phenyl)-methanone, 225 mg of 1-triisopropylsilylpyrrol-3-boronic acid, 30 mg of tetrakis(triphenyl-phosphine)palladium, 2 ml of a 2M solution of sodium carbonate, 4 ml of toluene and 4 ml of acetonitrile. The reaction mixture is heated for 20 minutes in a microwave apparatus set at 150° C., and then cooled, diluted with ethyl acetate, filtered through celite, dried over magnesium sulphate and concentrated under reduced pressure. The residue is chromatographed on a silica cartridge, elution being carried out with a mixture of dichloromethane and ethyl acetate (90/10). The fractions containing the expected product are combined and concentrated to dryness under reduced pressure, to give 110 mg of phenyl[6-(1-triisopropylsilylpyrrol-3-yl)imidazo[1,2-a]pyridin-2-yl]methanone in the form of a green oil.

¹H NMR spectrum (DMSO-d6, δ in ppm): 1.09 (d, J=7.5 Hz, 18H), 1.56 (m, 3H), 6.67 (dd, J=1.5 and 2.0 Hz, 1H), 6.94 (t, J=2.0 Hz, 1H), 7.40 (broad s, 1H), 7.58 (t, J=7.5 Hz, 2H), 7.67 (m, 2H), 7.78 (dd, J=1.5 and 9.5 Hz, 1H), 8.33 (broad d, J=7.5 Hz, 2H), 8.51 (s, 1H), 8.82 (broad s, 1H).

Mass spectrum (LC-MS-DAD-ELSD): m/z 444 [M+H]⁺.

### 3.2: Phenyl[6-(1H-pyrrol-3-yl)imidazo[1,2-a]pyridin-2-yl]methanone

A solution of 110 mg of phenyl[6-(1-triisopropylsilylpyrrol-3-yl)imidazo[1,2-a]pyridin-2-yl]methanone in 1.5 ml of tetrahydrofuran is treated with 248 μl of a 1M solution of tetrabutylammonium fluoride in tetrahydrofuran, stirred for 2 hours at 25° C. and then concentrated under reduced pressure. The residue is chromatographed on a silica cartridge, elution being carried out with a mixture of dichloromethane and ethyl acetate (75/25). The fractions containing the expected product are combined and concentrated to dryness under reduced pressure, to give 31 mg of phenyl[6-(1H-pyrrol-3-yl)imidazo[1,2-a]pyridin-2-yl]methanone in the form of a brown solid.

### EXAMPLE 4

### Phenyl[6-(1H-pyrazol-4-yl)imidazo[1,2-a]pyridin-2-yl]methanone

A microwave tube is loaded with 250 mg of (6-iodolimidazo[1,2-a]pyridin-2-yl)-(phenyl)methanone, 225 mg of 1H-4-pyrrazoleboronic acid, 33 mg of tetrakis(triphenylphosphine)-palladium, 2.5 ml of a 2M solution of sodium carbonate, 5 ml of toluene and 5 ml of acetonitrile. The reaction mixture is heated for 20 minutes in the microwave apparatus set at 150° C., and then cooled, diluted with ethyl acetate, filtered through celite, dried over magnesium sulphate and concentrated under reduced pressure. The residue is chromatographed on a silica cartridge, elution being carried out with ethyl acetate. The fractions containing the expected product are combined and concentrated to dryness under reduced pressure, to give 15 mg of phenyl[6-(1H-pyrazol-4-yl)imidazo[1,2-a]pyridin-2-yl]methanone in the form of a beige solid.

### EXAMPLE 5

### [6-(1H-Imidazol-4-yl)imidazo[1,2-a]pyridin-2-yl](phenyl)methanone

### 5.1: Phenyl[6-(1-trityl-1H-imidazol-4-yl)imidazo[1,2-a]pyridin-2-yl]methanone

A microwave tube is loaded with 200 mg of phenyl[6-(4,4,5,5-tetramethyl-1,3,2-dioxaborolan-2-yl)imidazo[1,2-a]pyridin-2-yl]methanone, 276 mg of 4-iodo-1-tritylimidazole, 26 mg of tetrakis(triphenylphosphine)palladium, 2 ml of a 2M solution of sodium carbonate, 4 ml of toluene and 4 ml of acetonitrile. The reaction mixture is heated for 20 minutes in the microwave apparatus set at 150° C., and then cooled, filtered through celite, diluted with ethyl acetate, dried over magnesium sulphate and concentrated under reduced pressure. The residue is chromatographed on a silica cartridge, elution being carried out with a mixture of dichloromethane and ethyl acetate (75/25). The fractions containing the expected product are combined and concentrated to dryness under reduced pressure. The solid is triturated in a mixture of methanol and pentane and then dried, to give 135 mg of phenyl[6-(1-trityl-1H-imidazol-4-yl)imidazo[1,2-a]pyridin-2-yl]methanone in the form of a pale yellow solid.

¹H NMR spectrum (DMSO-d6, δ in ppm): 7.19 (d, J=7.5 Hz, 6H), 7.7 to 7.48 (m, 9H), 7.51 to 7.60 (m, 4H), 7.65 (m, 2H), 7.79 (dd, J=1.5 and 9.5 Hz, 1H), 8.32 (d, J=7.5 Hz, 2H), 8.53 (s, 1H), 9.02 (broad s, 1H).

Mass spectrum (LC-MS-DAD-ELSD): m/z 531 [M+H]⁺.

### 5.2: [6-(1H-Imidazol-4-yl)imidazo[1,2-a]pyridin-2-yl](phenyl)methanone

A suspension of 133 mg of phenyl[6-(1-trityl-1H-imidazol-4-yl)imidazo[1,2-a]pyridin-2-yl]-methanone in a mixture of 5 ml of 2N hydrochloric acid and 1 ml of acetic acid is refluxed for 2.5 hours. The solid is filtered and washed with hot water. The filtrate is neutralized with potassium carbonate and extracted with dichloromethane. The organic phase is dried over magnesium sulphate and concentrated under reduced pressure. The solid is dried under reduced pressure, to give 58 mg of [6-(1H-imidazol-4-yl)imidazo[1,2-a]pyridin-2-yl](phenyl)methanone in the form of a beige solid.

### EXAMPLE 6

### (6-Furan-2-ylimidazo[1,2-a]pyridin-2-yl)(phenyl)methanone

A microwave tube is loaded with 250 mg of (6-iodolimidazo[1,2-a]pyridin-2-yl)-(phenyl)methanone, 843 mg of tributylfuran-2-ylstannane, 151 mg of tetrakis(triphenylphosphine)-palladium and 4 ml of N,N-dimethylformamide. The reaction mixture is heated for 10 minutes in the microwave apparatus set at 150° C., and then cooled and concentrated under reduced pressure. The residue is chromatographed on a silica cartridge, elution being carried out with a mixture of dichloromethane and ethyl acetate (90/10). The fractions containing the expected product are combined and concentrated to dryness under reduced pressure. The residue is crystallized from a mixture of methanol and diisopropyl ether, to give 124 mg of (6-furan-2-ylimidazo[1,2-a]pyridin-2-yl)(phenyl)methanone in the form of a beige solid.

The intermediates described below are useful in the preparation of the compounds of the present invention.

### Intermediate 1: (6-Iodoimidazo[1,2-a]pyridin-2-yl)(phenyl)methanone

A solution of 5.5 g of 3-bromo-1-phenylpropane-1,2-dione in 35 ml of tetrahydrofuran is added to a solution of 4.5 g of 2-amino-5-iodopyridine in 35 ml of tetrahydrofuran. The reaction mixture is stirred for 16 hours at 20° C. and then concentrated to dryness under reduced pressure. The residue is taken up in 100 ml of ethanol and refluxed for 2.5 hours. The reaction mixture is concentrated to dryness and taken up in a saturated solution of sodium bicarbonate and dichloromethane. The organic phase is dried over magnesium sulphate and evaporated to dryness. The solid is triturated in ethyl ether, filtered and dried, to give 4.14 g of (6-iodoimidazo[1,2-a]pyridin-2-yl)(phenyl)methanone in the form of an orangey-yellow solid.

¹H NMR spectrum (DMSO-d6, δ in ppm): 7.58 (t, J=7.6 Hz, 4H), 7.67 (d, J=7.3 Hz, 1H), 8.30 (d, J=7.8 Hz, 1H), 8.54 (s, 1H), 9.01 (s, 1H).

Mass spectrum (LC-MS-DAD-ELSD): m/z 349: [M+H]⁺.

Intermediate 1 ((6-iodoimidazo[1,2-a]pyridin-2-yl)(phenyl)methanone) is already described in document WO 2008/003854 (compound 22 of the table). In this respect, it is excluded from the scope of the present invention.

### Intermediate 2: Phenyl[6-(4,4,5,5-tetramethyl-1,3,2-dioxaborolan-2-yl)imidazo[1,2-a]-pyridin-2-yl]methanone

A solution of 6 g of 3-bromo-1-phenylpropane-1,2-dione in 50 ml of tetrahydrofuran is added to a solution of 7 g of 2-amino-5-(4,4,5,5-tetramethyl-1,3,2-dioxaborolan-2-yl)pyridine in 50 ml of tetrahydrofuran. The reaction mixture is stirred for 16 hours at ambient temperature and then concentrated to dryness under reduced pressure. The residue is taken up in 100 ml of ethanol and refluxed for 2.5 hours. After concentration to dryness under reduced pressure, the solid is taken up in a saturated solution of sodium bicarbonate and dichloromethane. The organic phase is dried over magnesium sulphate and evaporated to dryness. The residue is redissolved in 30 ml of ethanol and 12 ml of 2N hydrochloric acid. The reaction mixture is stirred at 25° C. and then evaporated to dryness and taken up in dichloromethane and 24 ml of 1N sodium hydroxide. The organic phase is dried over magnesium sulphate and evaporated to dryness. The residue is triturated in a mixture of dichloromethane and ethyl ether. The insoluble material is filtered off and the concentrated filtrate is triturated in a mixture of dichloromethane, methanol and ethyl ether. The solid is filtered off and dried, to give 1.2 g of phenyl[6-(4,4,5,5-tetramethyl-1,3,2-dioxaborolan-2-yl)imidazo[1,2-a]pyridin-2-yl]methanone in the form of an orangey-beige solid.

¹H NMR spectrum (DMSO-d6, δ in ppm): 1.33 (s, 12H), 7.44 (dd, J=1.5 and 9.5 Hz, 1H), 7.58 (t, J=7.5 Hz, 2H), 7.69 (m, 3H), 8.30 (d, J=7.5 Hz, 2H), 8.70 (s, 1H), 8.95 (broad s, 1H).

Mass spectrum (1E): m/z 348: [M]⁺.

The tables which follow illustrate the chemical structures (Table 1) and the spectroscopic characteristics (Table 2) of some examples of compounds according to the invention.

In the “salt” column of Table 1, “-” represents a compound in the form of a free base, whereas “HCl” represents a compound in hydrochloride form, and the ratio between parentheses is the (acid:base) ratio,

TABLE 1
(I)

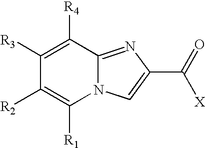

| Ex | R₁ | R₂ | R₃ | R₄ | X | Salt |
|---|---|---|---|---|---|---|
| 1 | H | 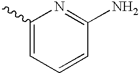 | H | H | 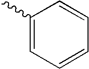 | — |
| 2 | H | 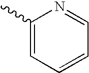 | H | H | 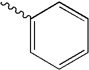 | HCl (2:1) |
| 3 | H | 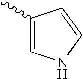 | H | H | 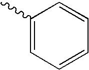 | — |
| 4 | H | 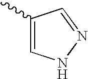 | H | H | 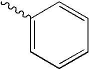 | — |
| 5 | H | 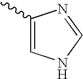 | H | H | 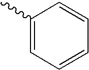 | — |
| 6 | H | 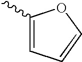 | H | H | 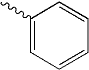 | — |
| 7 | H | 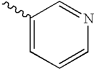 | H | H | 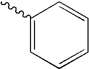 | — |

TABLE 2

| Compound | Characterizations |
|---|---|
| 1 | ¹H NMR spectrum (DMSO-d6, δ in ppm): 6.08 (broad s, 2 H), 6.49 (d, J = 7.8 Hz, 1 H), |
| | 7.10 (d, J = 7.8 Hz, 1H), 7.52 (t, J = 7.8 Hz, 1 H), 7.59 (t, J = 7.6 Hz, 2 H), 7.68 (t, J = 7.6 Hz, |
| | 1 H), 7.76 (d, J = 9.8 Hz, 1 H), 7.99 (dd, J = 9.8, 2.0 Hz, 1 H), 8.33 (broad d, J = 7.8 Hz, 2 |
| | H), 8.72 (s, 1 H), 9.20 (broad s, 1 H). |
| | Mass spectrum (LC-MS-DAD-ELSD): m/z 315 [M + H]⁺. |
| 2 | ¹H NMR spectrum (DMSO-d6, δ in ppm): 7.49 (broad dd, J = 5.0 and 80, Hz 1H), 7.64 |
| | (t, J = 7.5 Hz, 2H), 7.76 (t, J = 7.5 Hz, 1H), 7.94 (d, J = 9.5 Hz, 1H), 8.03 (dt, J = 1.5 and |
| | 8.0 Hz, 1H), 8.09 (d, J = 8.0 Hz, 1H), 8.22 (d, J = 7.5 Hz, 2H), 8.40 (broad d, J = 9.5 Hz, |
| | 1H), 8.75 (d, J = 5.0 Hz, 1H), 8.91 (s, 1H), 9.54 (broad s, 1H). |
| | Mass spectrum (LC-MS-DAD-ELSD): m/z 300 [M + H]⁺. |
| 3 | ¹H NMR spectrum (DMSO-d6, δ in ppm): 6.47 (dd, J = 1.5 and 2.0 Hz, 1H), 6.87 (dd, J = |
| | 1.0 and 2.0 Hz, 1H), 7.32 (dd, J = 1.0 and 1.5 Hz, 1H), 7.59 (t, J = 7.5 Hz, 2H), from |
| | 7.62 to 7.73 (m, 3H), 8.32 (d, J = 7.5 Hz, 2H), 8.53 (s, 1H), 8.77 (broad s, 1H), 11.05 |
| | (broad m, 1H). |
| | Mass spectrum (LC-MS-DAD-ELSD): m/z 286 [M − H]⁺, m/z 288 [M + H]⁺. |
| 4 | ¹H NMR spectrum (DMSO-d6, δ in ppm): 7.59 (t, J = 7.5 Hz, 2H), 7.69 (t, J = 7.5 Hz, |
| | 1H), 7.72 (m, 2H), 8.13 (broad m, 2H), 8.32 (d, J = 7.5 Hz, 2H), 8.53 (s, 1H), 8.91 |
| | (broad s, 1H), 13.05 (broad m, 1H). |
| | Mass spectrum (LC-MS-DAD-ELSD): m/z 287 [M − H]⁺, m/z 289 [M + H]⁺. |
| 5 | ¹H NMR spectrum (DMSO-d6, δ in ppm): 7.59 (t, J = 7.5 Hz, 2H), 7.68 (t, J = 7.5 Hz, |
| | 1H), 7.72 (m, 2H), 7.80 (m, 2H), 8.33 (d, J = 7.5 Hz, 2H), 8.68 (s, 1H), 9.00 (broad s, |
| | 1H), 12.3 (broad m, 1H). |
| | Mass spectrum (LC-MS-DAD-ELSD): m/z 287 [M − H]⁺, m/z 289 [M + H]⁺. |
| 6 | ¹H NMR spectrum (DMSO-d6, δ in ppm): 6.66 (dd, J = 3.4, 2.0 Hz, 1H) 7.07 (broad d, |
| | J = 3.4 Hz, 1H) 7.58 (t, J = 7.3 Hz, 2H) 7.68 (tt, J = 7.3, 1.4 Hz, 1H) 7.73-7.81 (m, 2H) 7.83 |
| | (broad d, J = 2.0 Hz, 1H) 8.33 (m, 2H) 8.69 (s, 1H) 8.98 (broad s, 1H). |
| | Mass spectrum (LC-MS-DAD-ELSD): m/z 289 [M + H]⁺. |
| 7 | ¹H NMR spectrum (DMSO-d6, δ in ppm): 7.56 (dd, J = 5.0 and 8.0 Hz, 1H), 7.60 (t, J = |
| | 7.5 Hz, 2H), 7.70 (tt, J = 1.5 and 7.5 Hz, 1H), 7.81 (dd, J = 1.5 and 9.5 Hz, 1H), 7.86 (d, J = |
| | 9.5 Hz, 1H), 8.16 (td, J = 1.0 and 8.0 Hz, 1H), 8.34 (broad d, J = 7.5 Hz, 2H), 8.53 (m, |
| | 2H), 8.98 (d, J = 2.0 Hz, 1H), 9.09 (broad s, 1H). |
| | Mass spectrum (LC-MS-DAD-ELSD): m/z 322 [M + Na]⁺. |

The compounds according to the invention were the subject of pharmacological tests for determining their modulatory effect on NOT.

Evaluation of the In Vitro Activity on N2A Cells

The activity of the compounds according to the invention was evaluated on a cell line (N2A) endogenously expressing the mouse Nurr1 receptor and stably transfected with the NOT binding response element (NBRE) coupled to the luciferase reporter gene. The EC₅₀ values are between 0.01 and 1000 nM. The tests were carried out according to the procedure described below.

The Neuro-2A cell line comes from a standard commercial source (ATCC). The Neuro-2A clone was obtained from a spontaneous tumour originating from a mouse A albino strain produced by R. J Klebe et al. This Neuro-2A line is subsequently stably transfected with 8NBRE-luciferase. The N2A-8NBRE cells are cultured at a confluence in 75 cm² culture flasks containing DMEM supplemented with 10% of foetal calf serum, 4.5 g/l of glucose and 0.4 mg/ml of geneticin. After one week of culture, the cells are recovered with 0.25% trypsin for 30 seconds, and then resuspended in DMEM without phenol red, containing 4.5 g/l of glucose and 10% of Hyclone defatted serum, and deposited in white, transparent-bottom, 96-well plates. The cells are deposited in a proportion of 60 000 per well in 75 μl for 24 hours before the addition of the products. The products are applied in 25 μl and incubated for a further 24 hours. On the day of the measurement, an equivalent volume (100 μl) of Steadylite, is added to each well, followed by a waiting period of 30 minutes in order to obtain complete lysis of the cells and maximum production of the signal. The plates are then measured in a microplate luminescence counter after having been sealed with an adhesive film. The products are prepared in the form of a stock solution at 10⁻² M, and then diluted in 100% of DMSO. Each product concentration is diluted beforehand in culture medium before incubation with the cells thus containing a final concentration of 0.625% of DMSO.

For example, compounds No. 1, 4 and 7 showed an EC₅₀ of 0.7, 0.5 and 0.5 nM, respectively.

It therefore appears that the compounds according to the invention have a NOT-modulating effect.

The compounds according to the invention can therefore be used for the preparation of medicaments for their therapeutic use in the treatment or prevention of diseases involving NOT receptors.

Thus, according to another of its aspects, a subject of the invention is medicaments which comprise a compound of formula (I), or an addition salt of the latter with a pharmaceutically acceptable acid.

These medicaments are of use in therapeutics, in particular in the treatment and prevention of neurodegenerative diseases such as, for example, Parkinson's disease, Alzheimer's disease, tauopathies (for example, progressive supranuclear palsy, frontotemporal dementia, corticobasal degeneration, Pick's disease); cerebral traumas such as ischaemia and cranial traumas and epilepsy; psychiatric diseases such as schizophrenia, depression, substance dependence, attention deficit hyperactivity disorders; inflammatory diseases of the central nervous system, such as multiple sclerosis, encephalitis, myelitis and encephalomyelitis and other inflammatory diseases such as vascular pathologies, atherosclerosis, joint inflammations, arthrosis, rheumatoid arthritis osteoarthritis, Crohn's disease, ulcerative colitis; allergic inflammatory diseases such as asthma, autoimmune diseases such as type 1 diabetes, lupus, scleroderma, Guillain-Barré syndrome, Addison's disease and other immunomediated diseases; osteoporosis; cancers.

These compounds could also be used as a treatment combined with stem cell transplantations and/or grafts.

Thus, a subject of the present invention is directed towards a compound of formula (I) as defined above, for the treatment of the abovementioned diseases and disorders.

According to another of its aspects, the present invention relates to pharmaceutical compositions comprising, as active ingredient, a compound according to the invention. These pharmaceutical compositions contain an effective dose of at least one compound according to the invention, or a pharmaceutically acceptable salt of said compound, and also at least one pharmaceutically acceptable excipient.

Said excipients are chosen, according to the pharmaceutical form and the method of administration desired, from the usual excipients which are known to those skilled in the art.

In the pharmaceutical compositions of the present invention for oral, sublingual, subcutaneous, intramuscular, intravenous, topical, local, intratracheal, intranasal, transdermal or rectal administration, the active ingredient of formula (I) above, or salt thereof, may be administered in unit administration form, as a mixture with conventional pharmaceutical excipients, to animals and to humans for the prophylaxis or the treatment of the disorders or diseases above.

The suitable unit administration forms comprise oral administration forms such as tablets, soft or hard gel capsules, powders, granules and oral solutions or suspensions, sublingual, buccal, intratracheal, intraocular and intranasal administration forms, forms for administration by inhalation, topical, transdermal, subcutaneous, intramuscular or intravenous administration forms, rectal administration forms, and implants. For topical administration, the compounds according to the invention may be used in creams, gels, ointments or lotions.

By way of example, a unit administration form of a compound according to the invention in tablet form may comprise the following components:

| | | |
|---|---|---|
| Compound according to the invention | 50.0 | mg |
| Mannitol | 223.75 | mg |
| Sodium croscarmellose | 6.0 | mg |
| Maize starch | 15.0 | mg |
| Hydroxypropylmethylcellulose | 2.25 | mg |
| Magnesium stearate | 3.0 | mg |

There may be particular cases where higher or lower dosages are appropriate; such dosages do not depart from the context of the invention. According to the usual practice, the dosage appropriate for each patient is determined by the physician according to the method of administration and the weight and response of said patient.

According to another of its aspects, the present invention also relates to a method for treating the pathologies indicated above, which comprises the administration, to a patient, of an effective dose of a compound according to the invention, or a pharmaceutically acceptable salt thereof.

What is claimed is:

1. A compound of formula (I): 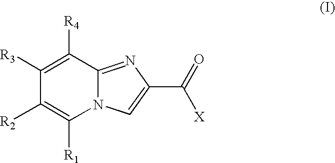
wherein:
X is a phenyl group optionally substituted with one or more atoms or groups chosen, independently of one another, from the following atoms or groups: halogen, (C₁-C₆)alkyl, (C₁-C₆)alkoxy, hydroxyl, amino, and NRaRb; the (C₁-C₆)alkyl and (C₁-C₆)alkoxy groups being optionally substituted with one or more halogen atoms;
R₂ is a heterocyclic group optionally substituted with one or more groups chosen, independently of one another, from the following atoms or groups: hydroxyl, halogen, (C₁-C₆)alkoxy, (C₁-C₆)alkyl optionally substituted with one or more hydroxyl, NRcRd, —CO—R₅, —CO—NR₆R₇, —CO—O—R₈, —NR₉—CO—R₁₀, cyano, and an oxido group;
R₁ is a hydrogen atom, a halogen, a (C₁-C₆)alkoxy group, a (C₁-C₆)alkyl group, hydroxyl or amino; it being possible for the (C₁-C₆)alkyl and (C₁-C₆)alkoxy groups to be optionally substituted with one or more of the following atoms or groups: halogen, hydroxyl, amino, and (C₁-C₆)alkoxy;
R₃ is a hydrogen atom, a halogen atom, a (C₁-C₆)alkyl group or a hydroxyl group;
R₄ is a hydrogen atom or a halogen atom;
R₅ is a hydrogen atom or a (C₁-C₆)alkyl group;
R₆ and R₇, which may be identical or different, are a hydrogen atom or a (C₁-C₆)alkyl group, or form, with the nitrogen atom which bears them, a 4- to 7-membered ring optionally including another heteroatom chosen from N, O or S;
R₈ is a (C₁-C₆)alkyl group;
R₉ and R₁₀, which may be identical or different, are a hydrogen atom or a (C₁-C₆)alkyl group;
Ra is a (C₁-C₆)alkyl; and
Rb, Rc and Rd are a hydrogen atom or a (C₁-C₆)alkyl;
or an acid addition salt thereof.

2. The compound of formula (I) according to claim 1, wherein:
X is a phenyl group;
R₁, R₃ and R₄ are hydrogen atoms; and
R₂ is an unsaturated monocyclic heterocyclic group containing 5 or 6 atoms, including from 1 to 2 heteroatoms chosen from N and O, said heterocyclic group being optionally substituted with an —NR_{c}R_{d} group, R_{c} and R_{d} being a hydrogen or a (C₁-C₆)alkyl;
or an acid addition salt thereof.

3. The compound of formula (I) according to claim 1, wherein:
X is a phenyl group;
R₁, R₃ and R₄ are hydrogen atoms; and
R₂ is a pyridine, pyrrole, pyrazole, imidazole or furan group, optionally substituted with an NH₂ group;
or an acid addition salt thereof.

4. The compound of formula (I) according to claim 1, selected from the group consisting of:
[6-(6-aminopyridin-2-yl)imidazo[1,2-a]pyridin-2-yl](phenyl)methanone,
phenyl(6-pyridin-2-ylimidazo[1,2-a]pyridin-2-yl)methanone and the dihydrochloride thereof,
phenyl[6-(1H-pyrrol-3-yl)imidazo[1,2-a]pyridine-2-yl]methanone,
phenyl[6-(1H-pyrazol-4-yl)imidazo[1,2-a]pyridin-2-yl]methanone,
[6-(1H-imidazol-4-yl)imidazo[1,2-a]pyridin-2-yl](phenyl)methanone,
[(6-furan-2-yl)imidazo[1,2-a]pyridin-2-yl](phenyl)methanone, and
phenyl[(6-pyridin-3-yl)imidazo[1,2-a]pyridin-2-yl]methanone,
or an addition salt thereof with a pharmaceutically acceptable acid.

5. A pharmaceutical composition comprising a compound of formula (I) according to claim 1, or a pharmaceutically acceptable salt thereof, and also a pharmaceutically acceptable excipient.

6. A pharmaceutical composition comprising a compound of formula (I) according to claim 2, or a pharmaceutically acceptable salt thereof, and also a pharmaceutically acceptable excipient.

7. A pharmaceutical composition comprising a compound of formula (I) according to claim 3, or a pharmaceutically acceptable salt thereof, and also a pharmaceutically acceptable excipient.

8. A pharmaceutical composition comprising a compound of formula (I) according to claim 4, or a pharmaceutically acceptable salt thereof, and also a pharmaceutically acceptable excipient.

9. A method of treating or preventing neurodegenerative diseases comprising administering to a patient an effective amount of a compound of formula (I) according to claim 1, or a pharmaceutically acceptable salt thereof.

10. A method of treating or preventing cerebral traumas or epilepsy comprising administering to a patient an effective amount of a compound of formula (I) according to claim 1, or a pharmaceutically acceptable salt thereof.

11. A method of treating or preventing psychiatric diseases comprising administering to a patient an effective amount of a compound of formula (I) according to claim 1, or a pharmaceutically acceptable salt thereof.

12. A method of treating or preventing inflammatory diseases comprising administering to a patient an effective amount of a compound of formula (I) according to claim 1, or a pharmaceutically acceptable salt thereof.

13. A method of treating or preventing osteoporosis or cancers comprising administering to a patient an effective amount of a compound of formula (I) according to claim 1, or a pharmaceutically acceptable salt thereof.

14. A method of treating or preventing Parkinson's disease, Alzheimer's disease, tauopathies or multiple sclerosis comprising administering to a patient an effective amount of a compound of formula (I) according to claim 1, or a pharmaceutically acceptable salt thereof.

15. A method of treating or preventing schizophrenia, depression, substance dependence or attention deficit hyperactivity disorders comprising administering to a patient an effective amount of a compound of formula (I) according to claim 1, or a pharmaceutically acceptable salt thereof.

16. The compound phenyl[6-(4,4,5,5-tetramethyl-1,3,2-dioxaborolan-2-yl)imidazo[1,2-a]pyridin-2-yl]methanone.
